(19) United States
(12) Patent Application Publication
(10) Pub. No.: US 2009/0248123 A1
(43) Pub. Date: Oct. 1, 2009

(54) Implantable Elongate Member

(76) Inventors: Susan Romao-Duarte Daniels, Kwazulli Natal (ZA); Mitchell-Luke Manning, West Sussex (GB)

Correspondence Address: GrayRobinson, P. A., 401 E. LAS OLAS BLVD, SUITE 1850, FORT LAUDERDALE, FL, 33301, US

(21) Appl. No.: 12/087,955

(22) PCT Filed: Jan. 18, 2007
(86) PCT No.: PCT/GB2007/000134

§ 371 Date: Dec. 4, 2008

(30) Foreign Application Priority Data
Jan. 18, 2006 (GB) 0600953.4

(51) Int. Cl. A61N 1/05; A61M 25/00; B23P 11/00

(52) U.S. Cl. 607/116; 604/264; 29/592.1

(57) ABSTRACT

The present invention relates to a lead (100) or catheter (202) having at least one member (116, 210) with interstices for facilitating the in-growth of tissue to anchor the lead (100) or catheter (202). The present invention also relates to a method of manufacturing a lead (100) or catheter (202). Furthermore, the present invention relates to a method of anchoring a lead (100) or catheter 9202) in a body.

### FIELD OF THE INVENTION

The present invention relates to an implantable elongate member, along with associated methods of use and manufacture. More particularly, the present invention relates to an implantable elongate member, such as a catheter or an electrical stimulation lead, having means for anchoring the elongate member in position.

### BACKGROUND TO THE INVENTION

Leads, catheters and the like may be implanted at peripheral sites in the body, for example across the base of the scull, and the chest wall. The distal end of such a lead or catheter is the therapeutic end and if it is not anchored, the distal tip of the lead or catheter may migrate away from the target treatment area. This migration has been witnessed as late as 18 months post implant.

Typically leads or catheters are anchored only proximal to the target treatment site and in many instances this anchor point is a substantial distance from the treatment site. Such anchors may be in the form of a number of different structural configurations. These methods are effective only in preventing the lead or catheter from general migration but not in preventing distal tip migration.

A system for anchoring a lead or catheter is described in the Applicant's earlier application US2005/0246006 and the contents of this application are incorporated herein by reference in their entirety. A suture loop is incorporated into the lead approximately 5 mm from the distal tip and approximately 25 mm from the most distal electrode. This distal portion of the lead allows for an incision at the position of the distal suture loop which falls outside of the treatment area. An incision as described is not always desirable or possible. An electrode lead 1 having a suture loop 3 provided at its distal tip is illustrated in FIG. 1.

It is further known from US2005/0273138 to provide a flexible anchor having two curved legs that cross in a single turning direction to form a loop, wherein the legs are adapted to penetrate tissue. The ends of the curved legs may be blunt or sharp. The anchor can assume different configurations such as a deployed configuration and a delivery configuration, and the anchor may switch between these different configurations. In operation, the anchor may be inserted into tissue by releasing the anchor from a delivery configuration so that the anchor self-expands into the deployed configuration, so that the legs of the anchor may penetrate the tissue in a curved pathway. This method may be problematic to explant and is a relatively complicated mechanism to manufacture.

It is also known to provide a catheter with a Dacron® collar to form a barrier to prevent infection. The collar is provided at the proximal end of the catheter, at the catheter exit site when the catheter has been implanted. The in-growth of tissue into the collar at this exit site prevents the non-sterile portion of the catheter causing infection. However, catheters of this type still rely on conventional anchoring techniques, such as suture loops, to secure the implanted portion in position and, therefore, they are subject to the limitations and problems outlined above.

The present invention seeks to ameliorate or solve the problems associated with known apparatus and the associated methods.

### SUMMARY OF THE INVENTION

According to a first aspect of the present invention, there is provided a lead or catheter comprising at least one member having interstices for facilitating the in-growth of tissue to anchor the lead or catheter.

The lead or catheter is intended to be implanted in a human or animal body. After the lead or catheter has been implanted, the tissue surrounding the at least one member will grow into the interstices over a period of time. This in-growth of tissue secures the lead or catheter in position and helps to prevent distal tip migration. Unlike a suture loop, it is not necessary to have access to the tissue around the at least one member to secure it in position. Thus, the present invention, at least in preferred embodiments, offers a practical, less invasive method of securing an elongate member such as an electrode lead or catheter than other previously suggested methods.

The at least one member may be provided anywhere along the length of a lead or catheter. For example, members may be located at several points along the lead or catheter. Alternatively, a single member may encapsulate the entire length of a lead or catheter; or the lead or catheter may be constructed from a material which encourages tissue in-growth. Preferably, however, the member or multiple members is/are provided proximal a first end of the lead or catheter to help prevent migration. In embodiments comprising a plurality of members, some or all of the members may be provided proximal said first end; or the members may be distributed along the length of the lead or catheter. The first end of the lead or catheter is preferably the end locatable remotely from the incision through which the lead or catheter is inserted, i.e. the distal end. The member(s) may be located within 100 mm; 50 mm; or 25 mm of the first end of the lead or catheter. Preferably the member or multiple members is/are located at said first end of the lead or catheter. The member or multiple members is/are most preferably provided at the distal end or tip of the lead or catheter for location inside the body at any point or points distal to the incision or orifice through which the lead or catheter is intended to be inserted in the body.

The or each member preferably extends along between 5 mm and 20 mm of the length of the lead or catheter. It is particularly preferred that the or each member extends along approximately 10 mm of the length of the lead or catheter. A member or multiple members of this length should provide adequate anchoring for the lead or catheter once it has been implanted.

The lead preferably has at least one electrode disposed along its length. The or each electrode preferably extends for around 20 mm or more along the length of the lead. The at least one electrode may be spaced at least 5 mm from any member and, preferably, is spaced between 5 mm and 30 mm from any member. The electrodes preferably each comprise a group of separate electrical contacts. The electrical contacts preferably each extend for between 2 mm and 5 mm along the length of the electrode lead. Furthermore, the electrical contacts are preferably spaced apart from one another along the length of the electrode lead by around 2 mm or more.

The catheter may be open at the first end. Preferably, however, at least one port is provided along the length of the catheter. In use, fluid may be delivered through the at least one port to the tissue around the catheter.

The electrode on a lead or port on a catheter closest to a member is preferably spaced at least 5 mm from a member and, more preferably, between 5 mm and 30 mm from a member. This arrangement helps to ensure that a member does not impede the delivery of electrical stimulation from an electrode or of fluid from a catheter port.

A second end of the lead or catheter preferably remains proximal the incision through which the lead or catheter is implanted into the body. The second end of the lead is intended for connection to a power source such as a fully implantable pulse generator and the second end of the catheter is intended for connection to a reservoir. A connector may be provided at said second end of the lead or catheter for connecting the lead to the pulse generator or the catheter to the reservoir.

The at least one member preferably has a microporous structure. The at least one member may be made of any material which facilitates the ingrowth of tissue. The at least one member typically has interstices to allow the in-growth of tissue. Preferably, the lead or catheter is made of a plastics material or most preferably a fluoropolymer such as Polytetrafluoroethylene (PTFE). The at least one member may be made of a Dacron® type material or most preferably an expanded form of fluoropolymer, such as expanded Polytetrafluoroethylene (ePTFE) which is designed to encourage tissue in-growth.

The at least one member may have a larger diameter than the remainder of the lead or catheter. Preferably, however, the diameter of the at least one member is substantially the same as the diameter of the remainder of the lead or catheter. This helps to ensure that the lead or catheter may be implanted using conventional techniques.

The at least one member may be a collar or collars provided around the lead or catheter; or an insert or inserts provided in the lead or catheter. Alternatively or additionally, the member may be a cap or cuff provided at the distal end of the lead or catheter.

The at least one member may be secured in position using adhesive or may be retained in position mechanically. For example, the at least one member may be integral to the lead or catheter or may be a restriction fit in the lead or catheter or may be threaded and screwed onto the lead or catheter. Preferably however the at least one member will be integral to the lead or catheter.

It will be appreciated that more than one member may be provided along the length of the lead or catheter for anchoring it in position. Furthermore, a suture loop or other anchor point may be provided at the proximal end of the lead or catheter and a member having interstices provided at the distal end thereof.

The present invention also relates to an alternate method of anchoring the distal end of the electrical nerve stimulation device comprising an electrode lead as described in the Applicant's earlier application US2005/0246006 and the contents of this application are incorporated herein by reference in their entirety. An electrical pulse generator may be connectable to the electrode lead for applying electrical potential across the electrical contacts. The proximal end of the lead is preferably attached to an electrical pulse generator and the at least one member provided at the distal end of the lead.

According to a further aspect, the present invention relates to a method of manufacturing an electrode lead or catheter, the method comprising the step of providing a member or multiple members on the lead or catheter, the member or multiple members being manufactured using a material which enables the in-growth of tissue. The member or members may be made of any suitable plastics material, but are preferably made of a Dacron® type material or an expanded fluoropolymer know as ePTFE.

According to a still further aspect, the present invention relates to a method of anchoring an electrode lead or catheter in a body, a portion or portions of the outside of the electrode lead or catheter having interstices; the method comprising implanting the electrode lead or catheter in the body; wherein the in-growth of tissue into the interstices anchors the electrode lead or catheter in position.

Viewed from a still further aspect, the present application relates to a lead or catheter implantable in a human or animal body, at least one region on the outside of the lead or catheter having interstices for facilitating the in-growth of tissue.

Viewed from a further aspect, the present invention relates to an elongate member implantable in a human or animal body, at least one region on the outside of the elongate member having interstices for facilitating the in-growth of tissue.

The at least one region having interstices preferably extend around the elongate member. The at least one region is preferably provided at an end of the elongate member. The at least one region is most preferably provided at the end of the elongate member intended to be located distal from the incision through which the elongate member is implanted into a body, i.e. the distal end of the elongate member or at any point distal to the incision through which the lead or catheter is inserted. This is desirable since migration of the distal tip of the elongate member may be prevented. The or each region preferably extends between 5 mm and 20 mm along the length of the elongate member.

The or each region having interstices may be formed integrally with the elongate member or may be formed as a separate component and attached to the elongate member.

The at least one region may be made of a material which facilitates the in-growth of fibrous tissue. Preferably, the at least one region is made of a plastics material. Most preferably, the at least one region is made of a Dacron® type material or an expanded fluoropolymer, such as ePTFE.

The diameter of the or each region is preferably substantially the same as, or slightly more than the diameter of the remainder of the elongate member. This helps to ensure that the elongate member may be implanted using conventional techniques.

The at least one region may be defined by a collar or multiple collars provided around the elongate member; or an insert or multiple inserts provided in the elongate member. Alternatively or additionally, the member may be a cap or cuff provided at the end of the elongate member. The at least one region may extend along substantially the entire length of the elongate member.

The elongate member is preferably made of PTFE.

The elongate member may be a catheter or a lead.

In embodiments in which the elongate member is a lead, one or more electrodes are preferably provided. The at least one region having interstices is preferably separate from or remote from said one or more electrodes. For example, a space or gap may be provided between the or each electrode and an adjacent region or regions having interstices. In embodiments in which the elongate member is a catheter, one or more ports are preferably provided. The at least one region having interstices is preferably separate from or remote from said one or more ports. For example, a space or gap may be provided between the or each port and an adjacent region or regions having interstices.

Viewed from a still further aspect, the present invention relates to method of manufacturing an elongate member, the method comprising the step of providing a member or multiple members having interstices for enabling the in-growth of tissue. The member or multiple members are preferably made of a Dacron® type material or an expanded fluoropolymer know as ePTFE.

Viewed from a yet further aspect, the present invention relates to a lead or catheter having a member or multiple members with interstices for facilitating the in-growth of tissue.

Viewed from a still further aspect, the present application relates to a catheter comprising a plurality of ports, wherein said ports are spaced apart along the length of the catheter. Catheters are conventionally provided with one or more ports at their distal end for releasing a fluid, such as an anaesthetic, into a localised area. The inventors in the present case have recognised the desirability of delivering a fluid to a series of different locations along the length of the catheter. This feature is considered to be patentable independently. The ports are preferably provided over a length of the catheter greater than or equal to 30 mm; 50 mm; 150 mm; 250 mm; 350 mm; or 450 mm. A Dacron® cuff or member or multiple members may be provided at the distal end of the catheter for anchoring it in place.

Viewed from a yet further aspect, the present invention relates to a lead or catheter having an outer surface with a microporous structure. The microporous structure may provide interstices to enable the in-growth of tissue to help anchor the lead or catheter in position. The lead or catheter may comprise an elongate member and the microporous structure may be formed integrally with said elongate member. Alternatively, an outer cover or sheath may be provided on the lead or catheter to provide the microporous structure.

Preferred embodiments of the present invention will now be described, by way of example only, with reference to the accompanying drawings.

### BRIEF DESCRIPTION OF THE DRAWINGS

FIG. 1 shows an electrode lead having a suture loop for anchoring its distal end;

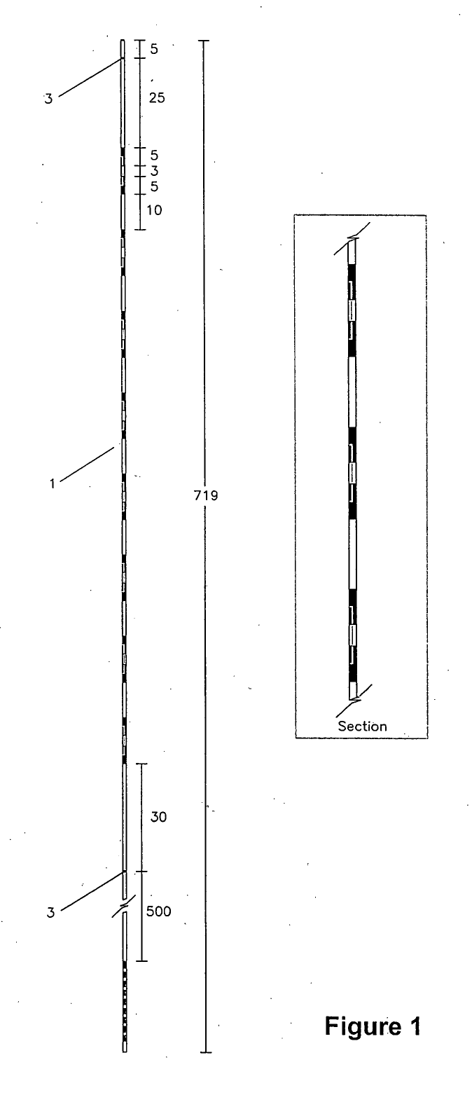

FIGS. 2a and 2b show an electrode lead in accordance with a first embodiment of the present invention;

FIG. 3 shows an electrode lead in accordance with a second embodiment of the present invention;

FIG. 4 shows an electrode lead in accordance with a third embodiment of the present invention;

FIG. 5 shows a catheter in accordance with a fourth embodiment of the present invention;

FIG. 6 shows a catheter in accordance with a fifth embodiment of the present invention; and

FIG. 7 shows a catheter in accordance with a sixth embodiment of the present invention.

### DETAILED DESCRIPTION OF THE PREFERRED EMBODIMENTS

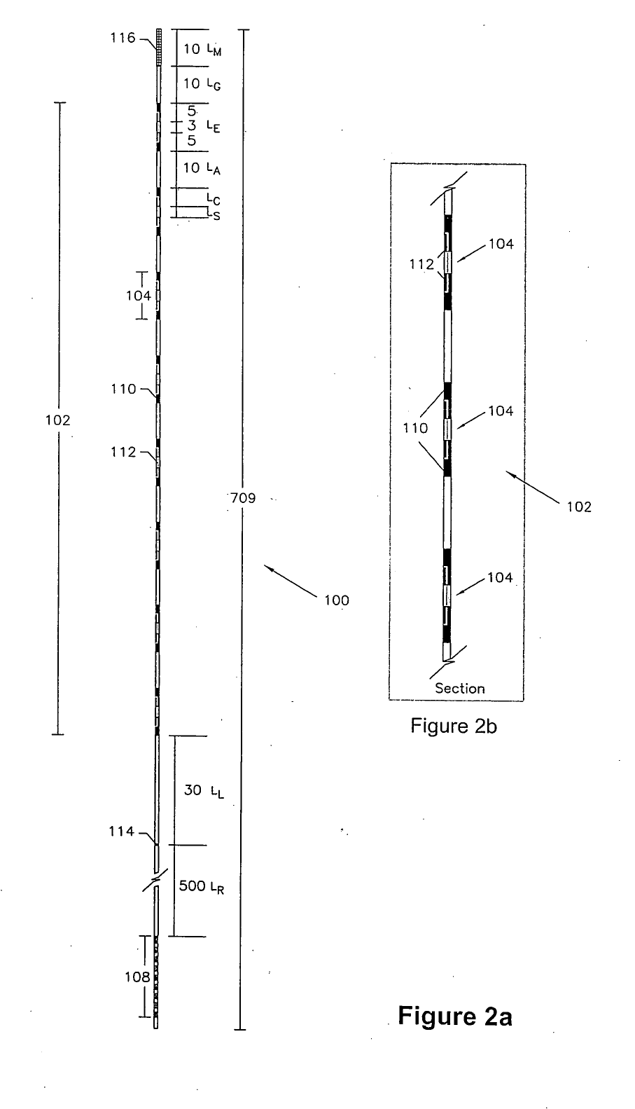

An electrode lead 100 in accordance with a first embodiment of the present invention is illustrated in FIGS. 2a and 2b. The electrode lead 100 is peripherally implantable in a human or animal body to provide electrical nerve stimulation.

The electrode lead 100 comprises an electrode array 102 of two or more electrodes 104 mounted on an elongate element 106 and a connector 108 positioned at one end of the elongate element 106. The lead 100 is made of a flexible, biocompatible, insulating material, such as polyurethane or polyethylene.

The electrodes 104 each comprise a series of contacts 110 joined to one another by wires 112 inside the elongate elements 106. So, whilst each electrode 104 has multiple contacts 110, it is effectively only a single “electrode” or “contact set”. The contacts 110 are made from a biocompatible conductor, such as a platinum/iridium alloy and are relatively solid and inflexible in that they extend around the respective leads 100, e.g. they are substantially annular. However, the wires 112 are stainless steel strands, so are generally flexible. This construction makes the electrodes 104 and hence the electrode lead 100 largely flexible.

The elongate elements 106 are hollow and one or more wires (not shown) extend along the inside of the elongate elements 106 to provide electrical connection between the electrodes 104 and the connector 108. The connector 108 allows the lead 100 to be connected to a power source which may be implanted in a body.

The elongate elements 106 are each between around 70 mm and 900 mm long and have a diameter of around one millimetre, e.g. in this embodiment approximately 1.25 mm, with the electrode arrays 102 extending along the length of the elements 106 for between around 70 mm and 450 mm. More specifically, the lead 100 has a length L from its connector 108 to the end of the lead 100 distal to the connector 108 of 709 mm; the electrodes 104 extend along the length of each lead 100 for an overall distance L_{E} of around 13 mm; the electrodes 104 are spaced apart from one another by a distance L_{A} of 10 mm; and the individual contacts 110 extend along the lead 100 for a distance L_{C} of 5 mm and are spaced apart from one another by a distance L_{S} of 3 mm. The cap 116 extends along the lead 100 for a distance L_{M} of 10 mm and is spaced from the distal electrode 104 by a distance L_{G} of 10 mm. The electrode 104 closest to the suture loop 114 is spaced along the lead 100 by a distance L_{L} of 30 mm. The suture loop 114 in turn is spaced along the lead 100 from the connector 108 by a distance L_{R} of 500 mm.

A suture point 114 is provided on the elongate element 106 for securing the lead 100 in a body. The suture point 114 in the present case is a suture loop and is provided between the electrode array 102 and the connector 108. In the present embodiment the suture point 114 is located 30 mm from the electrode 104 nearest the connector 108.

A cap 116 is provided at the end of the lead 100 distal from the connector 108. The cap 116 is made of a material having interstices to facilitate the in-growth of fibrous tissue to anchor the cap 116 in position and thereby to prevent migration of the distal tip of the lead. The cap 116 in the present embodiment is made of Dacron®, but any material which promotes the desired in-growth of fibrous tissue may be employed. The cap 116 extends along 10 mm of the length of the elongate element 106.

The lead 100 is implanted using an introducing instrument (not shown) comprising a needle with a length around the same as that of the portion of the corresponding lead 100 from the proximal suture point 114 to the distal tip and a manipulator at one end of the needle. In this embodiment, the needle is a standard 14 gauge Touhy needle which is intended to be introduced into the tissue including its stylet to prevent coring, once the stylet is removed the needle is hollow. The lead 100 can be inserted at a desired position in the body by passing it into the needle. Once the lead 100 is in place, the needle can be removed by withdrawing it from the body.

After the lead 100 has been implanted for a short period of time, the surrounding tissue will grow into the interstices provided in the cap 116. This in-growth of fibrous tissue secures the distal tip of the lead 100 in position and helps to prevent migration of the distal tip of the lead. Thus, the present invention at least in preferred embodiments offers a practical, less invasive method of securing the distal tip of the lead 100 to the surrounding tissue than other previously suggested methods.

To explant the lead 100, a firm tug on the lead 100 is usually sufficient to remove the cap 116 from the surrounding tissue. It will be appreciated that the suture point 114 should first be released using conventional techniques.

The electrode lead 100 in accordance with the present invention can be used to treat neuropathic pain by electrically stimulating the nerves and nerve endings contained within the subcutaneous tissue or in the vicinity of a major peripheral nerve in a range of locations around the body and arising from a multitude of different causes. Examples include: post mastectomy pain; neuropathic chest wall pain; chronic post surgical pain; complex regional pain syndrome (CRPS); neuropathic head, neck and facial pain; neuropathic foot pain; penile/scrotal/testicular pain; post inguinal hernia repair pain; neuropathic abdominal wall pain; neuropathic failed back surgery syndrome (FBSS); angina; migraine; post traumatic thoracic or cervical neuropathic pain; intrascapular pain; vulvadynia; and coccydynia.

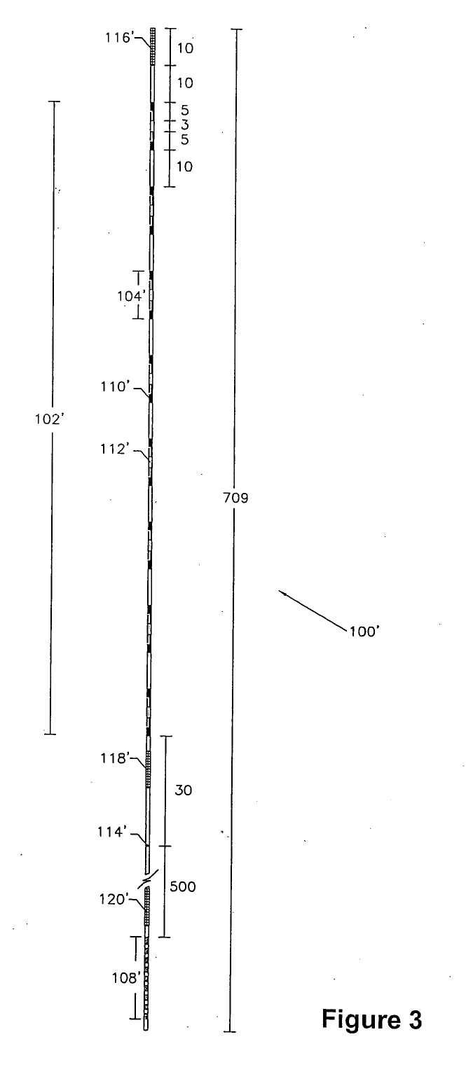

A lead 100′ in accordance with a second embodiment of the present invention is shown in FIG. 3. The lead 100′ is similar to the lead 100 according to the first embodiment described above and like reference numerals have been used for like components.

The lead 100′ is provided with first and second collars 118′, 120′ on each side of the suture loop 114′. The collars 118′, 120′ have a microporous structure providing interstices to allow the in-growth of tissue. The collars 118′, 120′ may be made of Dacron® or ePTFE. The collars 118′, 120′ may be formed integrally with the lead 100′ or they may formed separately and then attached to the lead 100′. In use, the in-growth of fibrous tissue into the interstices in the collars 118′, 120′ may help to anchor the lead 100′.

The cap 116′ is provided to anchor the distal tip of the lead 100′. However, in certain preferred embodiments, the cap 116′ may be omitted.

Furthermore, it will be appreciated that more than two collars 118′, 120′ may be provided along the length of the lead 100′.

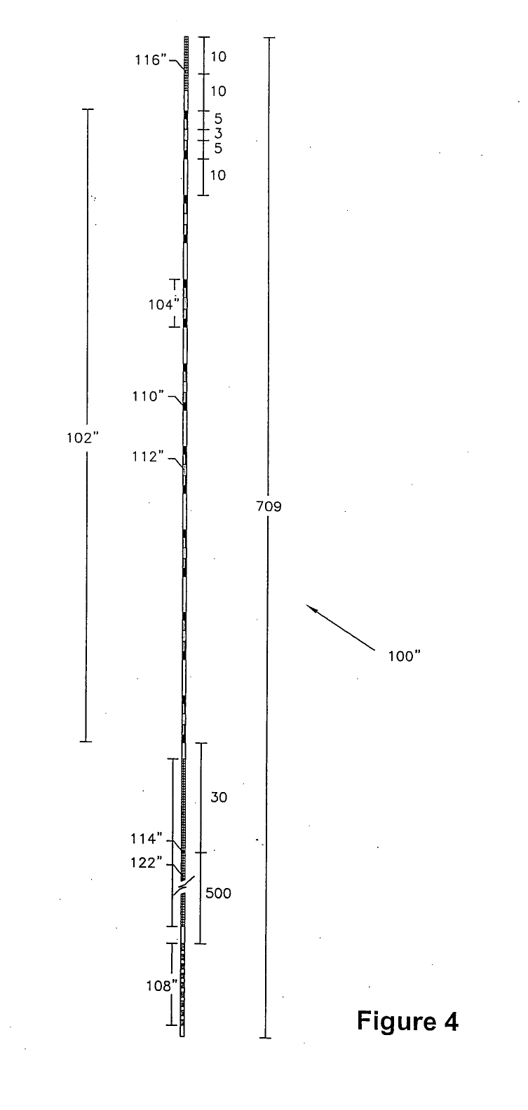

A lead 100″ in accordance with a third embodiment of the present invention is shown in FIG. 4. The lead 100″ is a modified version of the lead 100′ according to the second embodiment described above and like reference numerals have again been used for like components.

Rather than provide first and second collars 118′, 120′, a continuous region 122″ having a microporous structure is provided. The microporous structure provides interstices which allow the in-growth of fibrous tissues. The region 122″ extends along the lead 100″ and a suture loop 114″ is provided in the region 122″. The region 122″ may be made of Dacron® or ePTFE. The region 122″ may be formed integrally with the lead 100′ or separately, for example as a sheath or cover, and then attached to the lead 100′. In use, the in-growth of fibrous tissue into the interstices in the region 122″ may help to anchor the lead 100′.

The cap 116″ may be retained to anchor the distal tip of the lead 100″. However, in certain embodiments, the cap 116″ may be omitted.

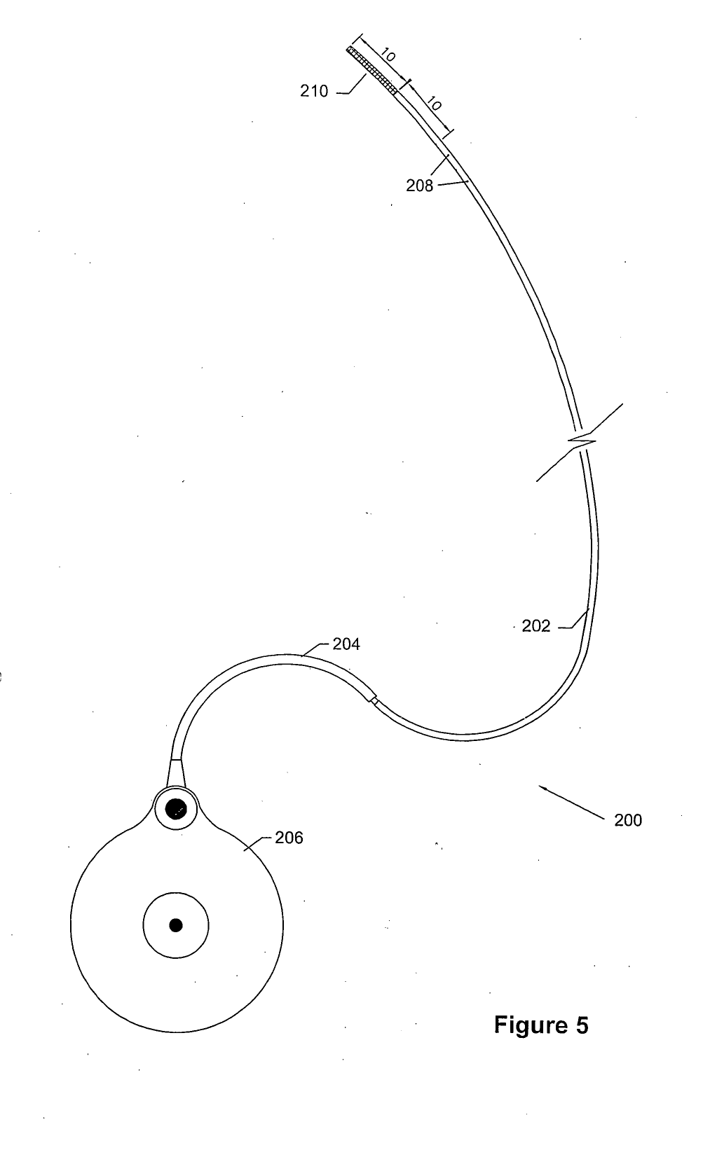

A device 200 for dispensing fluids into a body in accordance with a fourth embodiment of the present invention is illustrated in FIG. 5.

The device 200 comprises a catheter 202 and a connector 204 which are connected to a standard reservoir. The reservoir is sealed and a fluid, such as a local anaesthetic, is stored therein. The reservoir may be fully implantable in a body or it may form an external reservoir. The reservoir typically controls the dispensing of fluid into the catheter 202 in accordance with a predetermined treatment regime.

A series of ports 208 are provided along the catheter 202 to allow fluid to be dispensed from the reservoir. The location of the ports 208 along the length of the catheter 202 is selected to allow the fluid to be dispensed directly to selected target areas for localised treatment. The ports 208 in the present embodiment are provided near the end of the catheter 202 distal from the reservoir and are spaced apart from one another along the length of the catheter 202 by approximately 5 mm. The overall distance between the first and last port may be between 20 mm and 450 mm.

A cap 210 is provided at the end of the catheter 202 distal from the connector 204 to form a cuff. The cap 210 is made of a material having interstices which facilitates the in-growth of fibrous tissue to anchor the cap 210 in position and thereby to prevent migration of the distal tip of the catheter. The cap 210 in the present embodiment is made of Dacron®, but any material which facilitates or promotes the in-growth of fibrous tissue may be employed. The cap 210 extends along 10 mm of the length of the catheter 202. The cap 210 is spaced from the nearest port 208 by 10 mm along the length of the catheter 202.

The catheter 202 is implanted using known techniques. However, rather than employ a suture loop or the like to fix the tip of the catheter 202 to surrounding tissue, the cap 210 provides the required anchoring. After the catheter 202 has been implanted for a short period of time, fibrous tissue will grow into the interstices provided in the cap 210. This in-growth of fibrous tissue secures the tip of the catheter 202 in position and helps to prevent catheter tip migration. At least in preferred embodiments, the present invention offers a practical, less invasive method of securing the distal tip of a catheter to the surrounding tissue than other previously suggested methods.

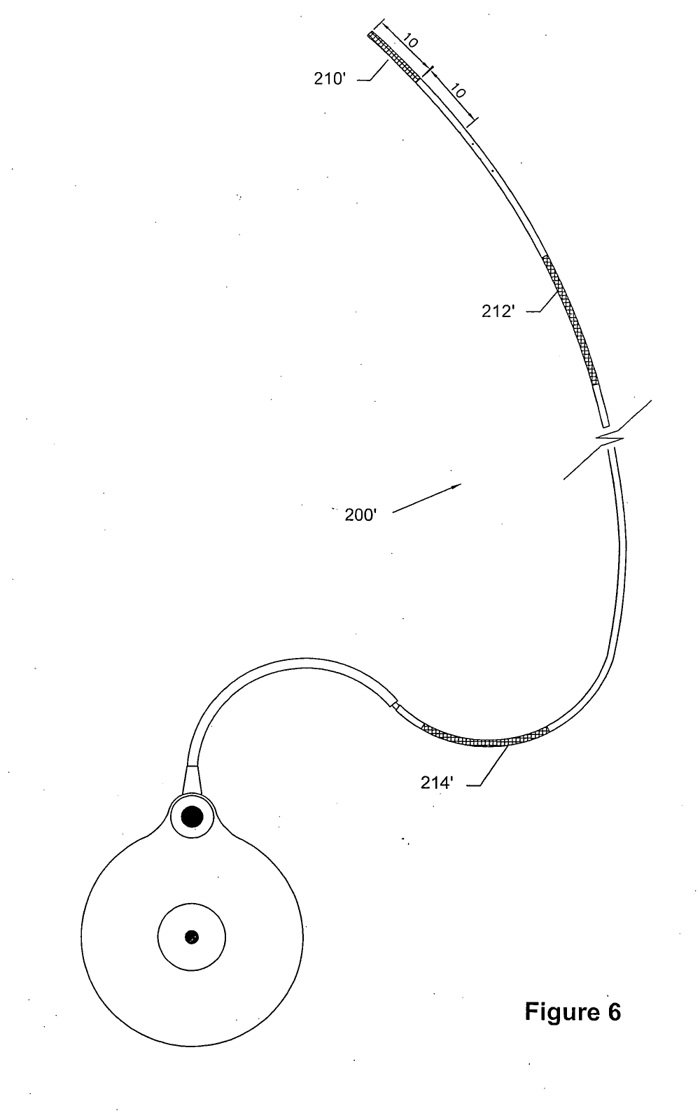

A catheter 200′ in accordance with a fifth embodiment of the present invention is shown in FIG. 6. The catheter 200′ is similar to the catheter 200 according to the fourth embodiment described above and like reference numerals have been used for like components.

The catheter 200′ is provided with first and second bands 212′, 214′ having a microporous structure. Interstices in the microporous structure allow the in-growth of tissue. The bands 212′, 214′ may be made of Dacron® or ePTFE. The bands 212′, 214′ may be formed integrally with the catheter 200′ or they may formed separately and then fixed in place. In use, the in-growth of fibrous tissue into the interstices may help to anchor the catheter 200′.

The cap 210′ may be retained to anchor the distal tip of the catheter 200′. However, in certain embodiments, the cap 210′ may be omitted.

Furthermore, it will be appreciated that more than two bands 212′, 214′ may be provided along the length of the catheter 200′.

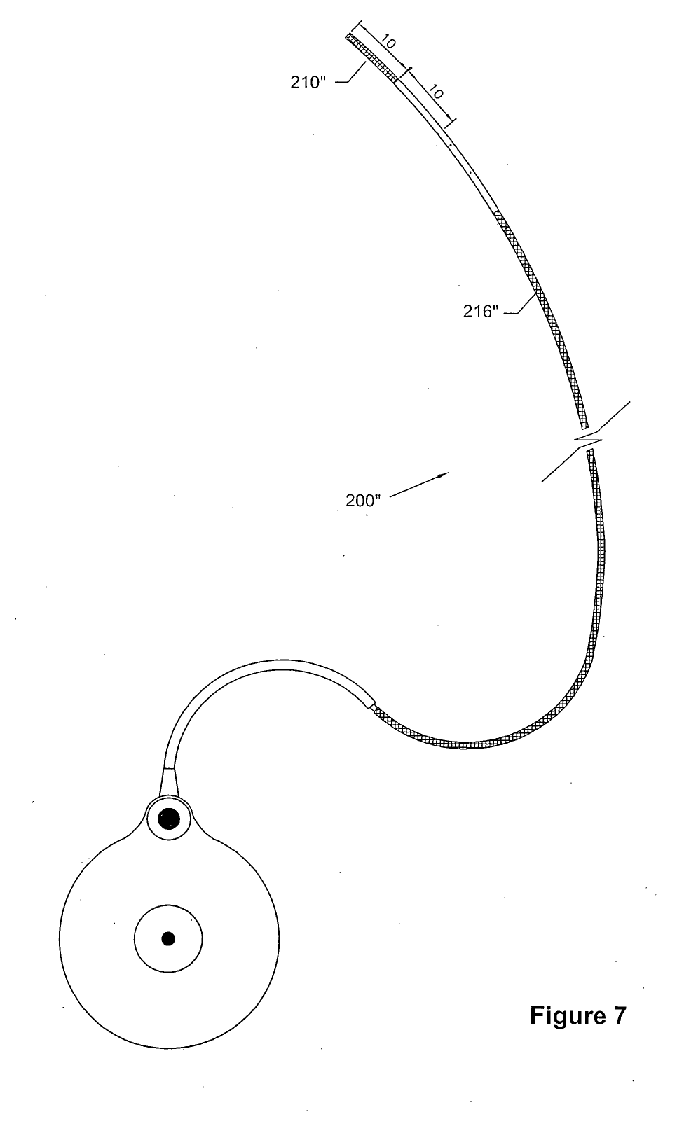

A catheter 200″ in accordance with a sixth embodiment of the present invention is shown in FIG. 7. The catheter 200″ is a modified version of the catheter 200′ according to the fifth embodiment described above and like reference numerals have again been used for like components.

Rather than provide first and second bands 212′, 214′, a continuous region 216″ having a microporous structure is provided along a portion of the length of the catheter 200″. Interstices in the microporous structure allow the in-growth of tissue. The region 216″ may be made of Dacron® or ePTFE. The region 216″ may be formed integrally with the catheter 200″ or separately, for example as a sheath or cover, and then fixed in place. In use, the in-growth of fibrous tissue into the interstices in the region 216″ may help to anchor the catheter 200″.

The cap 210″ may be retained to anchor the distal tip of the catheter 200″. However, in certain embodiments, the cap 210″ may be omitted.

The described embodiments of the invention are only examples of how the invention may be implemented. Modifications, variations and changes to the described embodiments will occur to those having appropriate skills and knowledge. These modifications, variations and changes may be made without departure from the spirit and scope of the invention defined in the claims and its equivalents.

For example, it will be appreciated that more than one member may be provided along the length of the electrode lead or catheter. Moreover, the invention is not limited to electrode leads and catheters and is applicable to any elongate member implantable in a human or animal body.

What is claimed is:

1. A lead or catheter having at least one member with interstices for facilitating the in-growth of tissue to anchor the lead or catheter.

2. A lead or catheter as claimed in claim 1, wherein the at least one member is provided proximal a first end of the lead or catheter.

3. A lead or catheter as claimed in claim 2, wherein the at least one member is located within 50 mm of said first end of the lead or catheter.

4. (canceled)

5. A lead or catheter as claimed in claim 2, wherein the at least one member is located at said first end of the lead or catheter.

6. A lead or catheter as claimed in claim 2, wherein said first end is the distal end of the lead or catheter.

7. A lead or catheter as claimed in claim 1, wherein the at least one member extends along between 5 mm and 20 mm of the length of the lead or catheter.

8. (canceled)

9. A lead or catheter as claimed in claim 1, wherein said at least one member is made of Dacron® or ePTFE.

10. A lead or catheter as claimed in claim 2, wherein the lead or catheter has a second end for connecting to a pulse generator or reservoir.

11. (canceled)

12. A lead or catheter as claimed in claim 10, wherein an anchor point is provided proximal said second end of the lead.

13. A lead or catheter as claimed in claim 1, further comprising at least one of an electrode and a port provided along a length of the lead or catheter.

14. A lead or catheter as claimed in claim 13, wherein the at least one of the electrode and the port is spaced between 5 mm and 30 mm from said at least one member.

15. A lead or catheter as claimed in claim 17, wherein a distance between a first electrical contact and a last electrical contact of the group of separate electrical contacts is greater than or equal to 2 mm.

16. A lead or catheter as claimed in claim 13, wherein the electrode extends for about 2 mm or more along the length of the lead or catheter.

17. A lead or catheter as claimed in claim 13, wherein the electrode comprises a group of separate electrical contacts.

18. A lead or catheter as claimed in claim 17, wherein each electrical contact of the group of separate electrical contacts extends for between 2 mm and 5 mm along the length of the lead.

19-20. (canceled)

21. A nerve stimulation device comprising a lead having at least one member with interstices for facilitating the in-growth of tissue to anchor the lead.

22. A lead or catheter as claimed in claim 1 further comprising a plurality of ports, wherein said plurality of ports are spaced apart along a length of the lead or catheter.

23. (canceled)

24. A lead or catheter implantable in a human or animal body, the lead or catheter comprising:
at least one outside region having interstices for facilitating the in-growth of tissue.

25. A lead or catheter as claimed in claim 24, wherein said at least one outside region extends around an exterior of the lead or catheter.

26. A lead or catheter as claimed in claim 24, wherein said at least one outside region is provided at a distal end of the lead or catheter.

27. A lead or catheter as claimed in claim 24, wherein the at least one outside or extends between 5 mm and 20 mm along a length of the lead or catheter.

28. A lead or catheter as claimed in claim 24, wherein the at least one outside region extends along substantially an entire length of the lead or catheter.

29. A lead or catheter as claimed in claim 25, wherein said at least one outside region is formed from Dacron® or ePTFE.

30. A method of manufacturing a lead or catheter, the method comprising the step of providing at least one member at a distal end of the lead or catheter, said at least one member having interstices for enabling an in-growth of tissue.

31. A method of manufacturing a lead or catheter as claimed in claim 30, wherein said at least one member is made of Dacron® or ePTFE.

32. A method of anchoring a lead or catheter in a body, a portion or multiple portions of the outside of the lead or catheter having interstices, the method comprising:
implanting the lead or catheter in the body; wherein an in-growth of tissue into the interstices anchors the lead or catheter in position.

33-34. (canceled)

35. A catheter comprising a plurality of ports, wherein said plurality of ports are spaced apart along a length of the catheter.

36. A catheter as claimed in claim 35, wherein the length of the catheter is greater than or equal to 30 mm.

37. A lead or catheter having an outer surface with a microporous structure.
